(19) United States
(12) Patent Application Publication
(10) Pub. No.: US 2003/0045510 A1
(43) Pub. Date: Mar. 6, 2003

(54) Estrogen-plus

(76) Inventors: Caroline Maxine Schloss, Knotts Island, NC (US); Dorothy Jean Fox, Chesapeake, VA (US)

Correspondence Address: CAROLINE M. SCHLOSS, DOROTHY J. FOX, P. O. Box 10, Knotts Island, NC, 27950, US

(21) Appl. No.: 09/736,944

(22) Filed: Dec. 15, 2000

(51) Int. Cl. A61K 31/56

(52) U.S. Cl. 514/171

(57) ABSTRACT

Estrogen-Plus will be a single pill 2 cm long, 1 cm wide, and about 0.5 cm thick, containing 1.25 to 2.5 mg of Estrogen, 100 mcg Selenium, 15 mg Zinc, 120 mcg Chromium, 150 mg Calcium, 2 mg Copper, 100 mg Phosphorus, 150 mg Magnesium, 75 mcg Molybdenum, 150 mcg Iodine, 5,000 IU Beta Carotene, 60 mg Ascorbic Acid, 400 IU Vitamin D, 30 IU Vitamin E, 80 mcg Vitamin K, 1.5 mg Thiamin, 1.7 mg Riboflavin, 2 mg Vitamin B6, 6 mcg Vitamin B12, 400 mcg Folic Acid, 18 mg Iron, 1 mg Pantothenic Acid, 300 mcg Biotin, and inert fillers and binders necessary for manufacture.

### BACKGROUND OF THE INVENTION

1. Field of the Invention

The present invention relates generally to a pharmaceutical supplement for menopausal women and more specifically to a pharmaceutical supplement which combines the hormone estrogen with daily supplemental vitamins to treat menopausal women and women who have undergone complete hysterectomies as more fully set forth in the below specifications, drawings and claims.

2. Description of Related Art

It is well known that estrogen is critical to a woman's health in that it helps to protect the cardiovascular system, helps protect against bone loss and aids mental sharpness. At menopause or subsequent to a complete hysterectomy, the estrogen levels decline significantly thus, the protective aspects of estrogen are significantly reduced for these women. Because heart disease is a major cause of death in women, this creates an increased risk for menopausal and post-hysterectomy women. Further, loss of the protection against bone loss can lead to osteoporosis, another major problem for these women. The impairment of cognitive abilities can be another side effect of the significant estrogen loss suffered in menopause or post-hysterectomy. Additional side effects have been linked to reduced estrogen levels such as urinary incontinence and weight gain.

Many women are treated with hormone replacement therapy to help reduce these symptoms. The treatment generally consists of supplemental estrogen. This reduces the problems noted above, heart disease, bone loss, loss of cognitive ability, urinary incontinence, weight gain, as well as other well-known symptoms such as hot flashes.

Numerous references teaching different hormone replacement systems exist. For example. U.S. Pat. No. 6,326,366 issued to Potter et al. teaches a hormone supplement formulation which minimizes the harmful side effects of hormone replacement therapy. U.S. Pat. No. 6,238,707 issued to Chun teaches an herbal hormone balance composition. U.S. Pat. No. 6,221,379 issued to Place teaches an improved delivery system for hormone replacement therapy. U.S. Pat. No. 5,516,528 issued to Hughes et al. provides a hormone replacement therapy in which soy derived phytoestrogens are utilized in combination with estrogen, instead of progestin. U.S. Pat. No. 5,468,736 issued to Hodgen is yet another formula for hormone replacement therapy which includes the administration of estrogen together with an amount of antiprogestin.

The present invention relates to two separate pharmaceutical compounds with the objective of their being combined to make one supplement having particular advantages for post-menopausal women and those who have had complete hysterectomies. The invention described herein has advantages over the related art of its field in that it is safe, separate previous arts are both approved by The Food And Drug Administration, easier to use, administer by virtue of consumption of one pill verses two, convenient, simple, economically constructed, obtainable through prescription, capable of serving the same function of the two separate pills by combining the two pills; hormone estrogen and daily supplemental vitamins into one form of pill which retains quality, quantity, size as well as providing doctor/medically recommended pharmaceutical, supplemental, health needs, for persons deemed as needing such.

Previous supplements for women exist. For example, U.S. Pat. No. 5,869,084 to Paradissis et al entitled Multi-Vitamin and Mineral Supplements for Women describes a nutritional supplement for women which is specially formulated for particular groups of women. The specific formulation for menopausal women includes vitamins and several minerals but does not teach the inclusion of estrogen.

### SUMMARY OF THE INVENTION

A multi-vitamin and hormone replacement supplement is provided for administration to menopausal women and women who have undergone hysterectomies. The supplement may include the following ingredients: Estrogen, Selenium, Zinc, Chromium, Calcium, Copper, Phosphorus, Magnesium, Molybdenum, Iodine, Beta Carotene, Ascorbic Acid, Vitamin D, Vitamin E, Vitamin K, Thiamin, Riboflavin, Vitamin B6, Vitamin B12, Folic Acid, Iron, Pantothenic Acid, and Biotin. The supplement may be administered daily or divided into portions and administered in portions during the day. The supplement may be in the form of a tablet, hard capsule, or soft gelatin capsule.

An object of the invention is to provide a multi-vitamin and hormone replacement supplement for administration to menopausal women and women who have undergone hysterectomies

Finally, it is an object of the present invention to accomplish the foregoing objectives in a simple and cost effective manner.

### BRIEF DESCRIPTION OF THE DRAWINGS

FIG. 1 represents an overhead view of the entire surface of the present invention in a pill form.

FIG. 2 represents a front and back view of this pill as identical.

FIG. 3 represents an isolated side view of this pill.

FIG. 4 represents an isolated view of the pill.

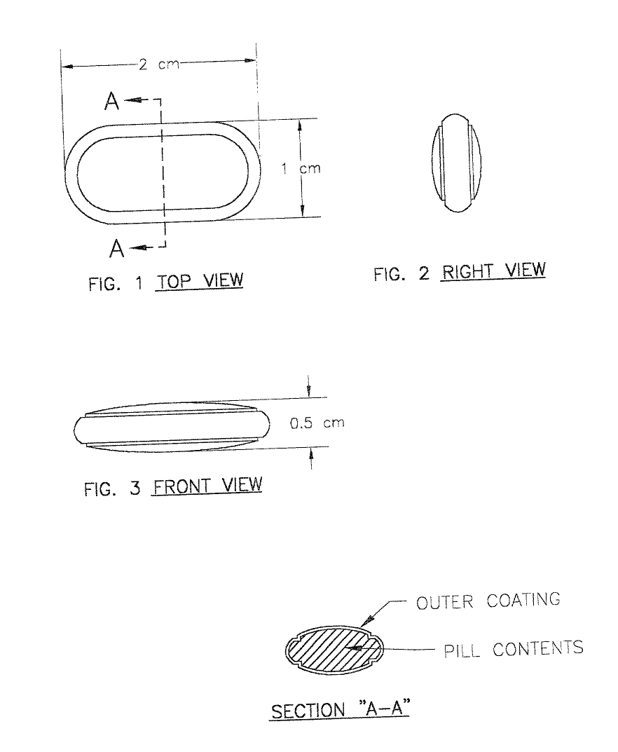

### DETAILED DESCRIPTION OF THE INVENTION

The following detailed description is of the best presently contemplated mode of carrying out the invention. This description is not to be taken in a limiting sense, but is made merely for the purpose of illustrating general principles of embodiments of the invention. Specifically, the amount ranges given are approximate.

A composition for treating menopausal women and women post-hysterectomy is as follows:

(a) 1.25 to 2.5 mg of Estrogen,

(b) 100 mcg Selenium,

(c) 15 mg Zinc,

(d) 120 mcg Chromium,

(e) 150 mg Calcium,

(f) 2 mg Copper,

(g) 100 mg Phosphorus,

(h) 150 mg Magnesium,

(i) 75 mcg Molybdenum,

(j) 150 mcg Iodine,

(k) 5,000 IU Beta Carotene,

(l) 60 mg Ascorbic Acid,

(m) 400 IU Vitamin D,

(n) 30 IU Vitamin E,

(o) 80 mcg Vitamin K,

(p) 1.5 mg Thiamin,

(q) 1.7 mg Riboflavin,

(r) 2 mg Vitamin B6,

(s) 6 mcg Vitamin B12,

(t) 400 mcg Folic Acid,

(u) 18 mg Iron,

(v) 1 mg Pantothenic Acid,

(w) 300 mcg Biotin,

and, as necessary, inert fillers and binders necessary for manufacture. As generally understood in the art, the amounts of each ingredient are approximate.

The onset of menopause or a complete hysterectomy causes a significant reduction in the estrogen level of women; the reduction can be as high as ninety percent. Estrogen, in conjunction with vitamin D and calcium, is critical to building and maintaining bone.

Selenium reduces the risk of heart attacks and heart disease, reduces the risk of cancer, protects against metal poisoning, and is synergistic with vitamin E.

Useful pharmaceutically acceptable zinc compounds include zinc sulfate, zinc chloride, and zinc oxide.

Chromium assists in the regulation of glucose metabolism, is used in the synthesis of fatty acids and cholesterol, assists in transporting proteins, lowers LDL blood levels, and raises high density lipoproteins blood levels. In this supplement, chromium is dosed in a pharmaceutically acceptable chromium compound. Useful pharmaceutically acceptable chromium compounds include, but are not limited to, yeast-bound chromium, GTF chromium, niacin-bound chromium.

Useful pharmaceutically acceptable calcium compounds include any of the well-known calcium supplements, such as calcium carbonate, calcium phosphate, calcium citrate, calcium sulfate, calcium oxide, calcium hydroxide, calcium apatite, calcium citrate-maleate, calcium lactate, calcium levulinate and the like.

Pharmaceutically acceptable copper compounds include cupric oxide, cupric sulfate, or cupric gluconate, with cupric oxide being preferred.

Along with the B vitamins, phosphorus is needed to extract energy from food, particularly fats and starches. It is a component of healthy bones, teeth, gums and many other tissues. Phosphorus also helps with kidney functioning and heart regularity. It lessens arthritis pain. Proper functioning of the phosphorus is only possible with the correct levels of vitamin D and calcium.

Magnesium is used in bone formation and growth, prevents bone loss, relaxes coronary arteries, is used in managing pre-eclampsia, treating cardiac arrhythmias, and managing diabetes. In this supplement, magnesium is dosed in the form of a pharmaceutically acceptable magnesium compound. Useful pharmaceutically acceptable magnesium compounds include, but are not limited to, magnesium stearate, magnesium carbonate, magnesium oxide, magnesium hydroxide, magnesium sulfate, and combinations thereof.

Molybdenum metabolizes fats and plays a biochemical role in the functioning of enzymes. Molybdenum also plays a role in iron utilization.

Iodine helps to metabolize fats, is necessary for proper thyroid function, and reduces fibrocystic breast conditions. In this supplement, iodine is dosed in the form of a pharmaceutically acceptable iodine compound. Useful pharmaceutically acceptable iodine compounds include, but are not limited to, potassium iodide, sodium iodide, and combinations thereof.

Beta Carotene is necessary for growth and repair of body tissues; helps maintain smooth, soft disease-free skin; helps protect the mucous membranes of the mouth, nose, throat & lungs, thereby reducing susceptibility to infections; protects against air pollutants; counteracts night-blindness & weak eyesight; aids in bone and teeth formation. Current medical research shows that foods rich in beta carotene will help reduce the risk of lung cancer & certain oral cancers.

Ascorbic Acid, also known as Vitamin C, is necessary for the synthesis of collagen and is used as an antioxidant. Vitamin C fights infection, reduces inflammation, heals wounds, reduces the risk of heart disease, lowers cholesterol, reduces the risk of lung, stomach, and esophageal cancers, reduces cervical epithelial abnormalities, inhibits N-nitrosamine and reduces the severity of colds.

Vitamin D is also an essential vitamin that is included in the supplement of the present invention. Vitamin D assists in the mineralization and calcification of bone, prevents rickets in children, prevents osteomalacia in adults, preserves bone and tooth growth, and lowers blood pressure. Vitamin D is fat soluble.

Vitamin E is needed for the maintenance of cell membranes and for neurological health. Vitamin E relieves hot flashes, relieves mastodynia, helps in fighting fibrocystic breast disease, reduces mammary tumors, reduces the risk of lung cancer, and reduces the risk of heart disease. Vitamin E is the generic term for a group of related substances which include alpha-tocopherol, beta-tocopherol, gamma-tocopherol, and delta-tocopherol. In addition, each of these four compounds have a “d” form, which is the natural form, and a “dl” form which is the synthetic form.

The supplement includes vitamin K,.an active blood clotting agent which assists in bone formation.

Thiamin or Vitamin B1 helps keep collagen-rich connective and mucous membranes healthy, helps to maintain smooth muscles, helps in the formation of blood cells, and is necessary for proper nervous system function.

Riboflavin or Vitamin B2 is necessary for healthy hair, nails, and mucous membranes and is involved in red blood cell formation, antibody production, and overall growth.

Vitamin B6 or pyridoxine is involved in the production of ribonucleic acid (RNA) and deoxyribonucleic acid (DNA) and many other reactions in the body. Pyridoxine refers to and includes three different compounds: pyridoxine, pyridoxamine, and pyridoxal.

Vitamin B12 or the cobalamins is necessary for overall metabolism, the function of the nervous system, metabolism of folic acid, and the production of red blood cells. There are at least three active forms of cobalamin: cyanocobalamin, hydroxocobalamin, and nitrocobalamin.

Folic acid is essential in the production of red blood cells, the production of hormones, and the synthesis of DNA.

The pharmaceutically acceptable iron compound may be chosen from any of the well-known iron II (ferrous) or iron III (ferric) supplements, such as ferrous fumarate, ferrous sulfate, carbonyl iron, ferrous glucomate, ferrous chloride, ferrous lactate, ferrous tartrate, ferrous succinate, ferrous glutamate, ferrous citrate, ferrous pyrophosphate, ferrous cholinisocitrate, ferrous carbonate, iron-sugar-carboxylate complexes and the like.

Pantothenic Acid is important for the production of adrenal gland hormones, increases overall energy, and helps convert food into energy.

Biotin is necessary for the metabolism of carbohydrates, proteins, and fats and is needed for healthy skin and hair.

When preparing dosage forms incorporating the compositions of the invention, the nutritional (components are normally blended with conventional excipients such as binders, including gelatin, pregelatinized starch, and the like; lubricants, such as hydrogenated vegetable oil, stearic acid, and the like; diluents, such as lactose, mannose, and sucrose; disintegrants, such as carboxymethyl cellulose and sodium starch glycolate; suspending agents, such as povidone, polyvinyl alcohol and the like; absorbent, such as silicon dioxide preservatives, such as methylparaben, propylparaben, and sodium benzoate surfactants, such as sodium lauryl sulfate, polysorbate 80, and the like; and colorants, such as F.D. & C dyes and lakes.

Many improvements, modifications, and additions will be apparent to the skilled artisan without departing from the spirit and scope of the present invention as described herein and defined in the following claims.

What is claimed is:

2 A multi-vitamin and hormone replacement supplement for administration to menopausal women and women who have undergone hysterectomies, which comprises:
(a) about 1.25 to 2.5 mg of Estrogen,
(b) about 150 mg Calcium, and
(c) about 400 IU Vitamin D.

3. The multi-vitamin and hormone replacement supplement as set forth in claim 2, which further comprises about 18 mg Iron.

4. The multi-vitamin and hormone replacement supplement as set forth in claim 2, which is administered orally once per day.

5. The multi-vitamin and hormone replacement supplement as set forth in claim 2, wherein the supplement is divided and administered in portions during the day.

6. The multi-vitamin and hormone replacement supplement as set forth in claim 2, wherein the supplement is in the form of a tablet.

7. The multi-vitamin and hormone replacement supplement as set forth in claim 2, wherein the supplement is filled into a hard capsule.

8. The multi-vitamin and hormone replacement supplement as set forth in claim 2, wherein the supplement is filled into a soft gelatin capsule.

9. A multi-vitamin and hormone replacement supplement for administration to menopausal women and women who have undergone hysterectomies, which comprises:
(a) about 1.25 to 2.5 mg of Estrogen,
(b) about 100 mcg Selenium,
(c) about 15 mg Zinc,
(d) about 120 mcg Chromium,
(e) about 150 mg Calcium,
(f) about 2 mg Copper,
(g) about 100 mg Phosphorus,
(h) about 150 mg Magnesium,
(i) about 75 mcg Molybdenum,
(j) about 150 mcg Iodine,
(k) about 5,000 IU Beta Carotene,
(l) about 60 mg Ascorbic Acid,
(m) about 400 IU Vitamin D,
(n) about 30 IU Vitamin E,
(o) about 80 mcg Vitamin K,
(p) about 1.5 mg Thiamin,
(q) about 1.7 mg Riboflavin,
(r) about 2 mg Vitamin B6,
(s) about 6 mcg Vitamin B12,
(t) about 400 mcg Folic Acid,
(u) about 1 mg Pantothenic Acid, and
(v) about 300 mcg Biotin.

10. The multi-vitamin and hormone replacement supplement as set forth in claim 9, which further comprises about 18 mg Iron.

11. The multi-vitamin and hormone replacement supplement as set forth in claim 9, which is administered orally once per day.

12. The multi-vitamin and hormone replacement supplement as set forth in claim 9, wherein the supplement is divided and administered in portions during the day.

13. The multi-vitamin and hormone replacement supplement as set forth in claim 9, wherein the supplement is in the form of a tablet.

14. The multi-vitamin and hormone replacement supplement as set forth in claim 9, wherein the supplement is filled into a hard capsule.

15. The multi-vitamin and hormone replacement supplement as set forth in claim 9, wherein the supplement is filled into a soft gelatin capsule.

16. A multi-vitamin and hormone replacement supplement for administration to menopausal women and women who have undergone hysterectomies, which comprises:
(a) about 1.25 to 2.5 mg of Estrogen,
(b) about 100 mcg Selenium,
(c) about 15 mg Zinc,
(d) about 120 mcg Chromium,
(e) about 150 mg Calcium,
(f) about 2 mg Copper,
(g) about 100 mg Phosphorus,
(h) about 150 mg Magnesium,
(i) about 75 mcg Molybdenum,
(j) about 150 mcg Iodine,
(k) about 5,000 IU Beta Carotene,
(l) about 60 mg Ascorbic Acid,
(m) about 400 IU Vitamin D,
(n) about 30 IU Vitamin E,
(o) about 80 mcg Vitamin K,
(p) about 1.5 mg Thiamin,
(q) about 1.7 mg Riboflavin,
(r) about 2 mg Vitamin B6,
(s) about 6 mcg Vitamin B12,
(t) about 400 mcg Folic Acid,
(u) about 18 mg Iron,
(v) about 1 mg Pantothenic Acid, and
(w) about 300 mcg Biotin.

17. The multi-vitamin and hormone replacement supplement as set forth in claim 16, which is administered orally once per day.

18. The multi-vitamin and hormone replacement supplement as set forth in claim 16 wherein the supplement is divided and administered in portions during the day.

19. The multi-vitamin and hormone replacement supplement as set forth in claim 16 wherein the supplement is in the form of a tablet.

20. The multi-vitamin and hormone replacement supplement as set forth in claim 16, wherein the supplement is filled into a hard capsule.

21. The multi-vitamin and hormone replacement supplement as set forth in claim 16, wherein the supplement is filled into a soft gelatin capsule.
